(19) United States
(12) Patent Application Publication
(10) Pub. No.: US 2003/0147972 A1
(43) Pub. Date: Aug. 7, 2003

(54) Method and composition for diminishing loss of color in flavors and fragrances

(76) Inventors: W. Denver Christopher, Perry Hall, MD (US); Richard Pisano JR., Cold Spring Harbor, NY (US)

Correspondence Address: Dennis P. Clarke, Miles & Stockbridge, Suite 500, 1751 Pinnacle Drive, McLean, VA, 22102, US

(21) Appl. No.: 10/062,561

(22) Filed: Feb. 5, 2002

(51) Int. Cl. A61K 35/78

(52) U.S. Cl. 424/725; 424/745

(57) ABSTRACT

A method and composition for stabilizing fragrance or flavor compositions against color-degradation based upon the use of an effective amount of a natural antioxidant that is substantially inert with respect to the fragrance compound, flavor compound or mixture thereof.

### BACKGROUND OF THE INVENTION

1. Field of the Invention

This invention relates to a method for preventing or diminishing the loss of color in products produced by the food, fragrance and flavor (F&F) industry.

2. Description of the Prior Art

Citrus oils and other materials employed in the F & F industry, such as paprika, tumeric, etc., often contain color components that are highly sensitive to light. Upon prolonged exposure to light irradiation, as well as other types of electromagnetic radiation, these materials fade in color strength or, in some cases, change color over time. As a result, these color-faded materials are unacceptable for use in the consumer products in which they were designed to be used. These destructive reactions are highly disruptive to the shelf life of the produced materials as it can cause long and short term problems for the food, flavor, or fragrance company producing the consumer products.

It is known from U.S. patent application Ser. No. 09/978,779, filed Oct. 18, 2001; hereinafter, the “application” (the entire contents and disclosures of which are incorporated herein by reference), that peroxides that are formed in F&F products when the latter auto-oxidize can cause problematic reactions in humans, either of an allergenic or sensitization nature, when the products are utilized in foods, cosmetics and the like. These reactions can be highly disruptive to the life of the sufferer and can have long and short term effects on the health of individuals exposed thereto. In the application, certain oxidative inhibitors were disclosed that would be acceptable for use around the world as antioxidant systems and which would not impart significant odor or flavor profiles to the material in which they were incorporated for stabilization purposes.

It is an object of the present invention to provide color-stabilization systems that will preserve the color characteristics of food, flavor and fragrance materials commonly employed in the F & F industry against the deleterious effects of electromagnetic irradiation, particularly light.

### SUMMARY OF THE INVENTION

The above and other objects are realized by the present invention, one embodiment of which relates to a fragrance or flavor composition comprising a fragrance effective amount of a fragrance compound, a flavor effective amount of a flavor compound or mixture thereof and an amount of a natural antioxidant that is substantially inert with respect to the fragrance compound, flavor compound or mixture thereof in an amount sufficient to substantially stabilize the fragrance or flavor composition against color degradation.

Another embodiment of the invention comprises a method for stabilizing a fragrance or flavor composition against color degradation, the fragrance or flavor composition comprising a fragrance effective amount of a fragrance compound, a flavor effective amount of a flavor compound or mixture thereof, the method comprising the step of incorporating a stabilizing amount of a natural antioxidant in the composition.

A still further embodiment of the invention concerns a method of using a fragrance or flavor composition, the fragrance or flavor composition comprising a fragrance effective amount of a fragrance compound, a flavor effective amount of a flavor compound or mixture thereof and an amount of a natural antioxidant that is substantially inert with respect to the fragrance compound, flavor compound or mixture thereof in an amount sufficient to substantially stabilize the fragrance or flavor composition against color degradation, comprising forming a composition containing an effective amount of the composition with a suitable carrier.

Another embodiment of the invention relates to an article of manufacture comprising a carrier having a fragrance or flavor composition incorporated therein wherein the fragrance or flavor composition comprises a fragrance effective amount of a fragrance compound, a flavor effective amount of a flavor compound or mixture thereof and an amount of a natural antioxidant that is substantially inert with respect to the fragrance compound, flavor compound or mixture thereof in an amount sufficient to substantially stabilize the fragrance or flavor composition against color degradation.

A final embodiment of the invention concerns articles of manufacture comprising cosmetic products, foods (including prepared foods, meats and the like), seasoning and flavoring blends that include effective amounts of one of the above-described compositions.

### DETAILED DESCRIPTION OF THE INVENTION

Advantages associated with the present invention include:

1) Enables the storage of materials without or less refrigeration than heretofore.

2) Non-obvious over synthetic color-stabilizers in that latter are prone to be reactive with the products to be stabilized.

The invention is predicated on the discovery that the utilization as color-stabilizers the materials found in the Labiatae family of plants, primarily rosemary, effectively stabilize the natural colors of the materials when incorporated in F&F materials. Moreover, the color-stabilizers function effectively without significantly altering the flavor and/or fragrance properties of the F&F materials. In a typical operation, the color-stabilizers materials are extracted from the plant and placed in a form which is conducive to introduction into F&F products. This invention makes use of, but not limited to, the compounds found in, e.g., rosemary: camosic acid, carnasol, rosmanol, rosmadial, and rosmarinic acid. With the addition of this extracted material via a carrier material to the finished F&F the onset of color-degradation is eliminated or delayed significantly.

In accordance with this invention, a typical F&F product will have added to it an amount of the rosemary (or other Labiatae extract) in the parts per billion (ppb) or parts per million (ppm) range. The amount added will vary depending upon the sensitivity of the F&F material to color-destabilization. The extract is preferably incorporated in the F&F product via a carrier material. The carrier material preferably comprises the same material as that to be stabilized. For example, to protect orange oil, the carrier employed would be orange oil. The color-stabilizer material and orange oil would be combined to form masterbatches of known concentrations. Portions of the masterbatches would then be combined with the F&F material to form the finished product having the desired proportions of ingredients.

It will be understood by those skilled in the art that any suitable method for combining the color-stabilizer material with the F&F material may be employed. For purposes of illustrating the invention herein, the direct dilution method will be described. It has been discovered that by adding a carrier material that contains the rosemary extract in predetermined concentrations to an F&F product the latter will be protected against color-degradation. [Methods for the detection of peroxides include titrametric utilizing the Peroxide value method found on page 198 of the Food Chemical Codex, 4th Edition.]

It is most preferred that the color-stabilizer material contains more than one of the stabilizing materials found in the plant material inasmuch as the color-stabilizer material are found to have a positive synergistic effect on each other if they are present in combination. Suitable flavor, food and fragrance materials that can be color-stabilized according to the invention include: citral, p-cymene, borneol, cinnamic alcohol, cinnamic aldehyde, dimethyl benzyl carbinol, 1-menthol, fenchyl alcohol, phenyl ethyl alcohol, o-tert-butyl cyclohexanol, 2-sec-butyl cyclohexanol, lauryl alcohol, 2-methyl undecanol, hexyl alcohol, citronellol, dihydro myrcenol, iso-amyl alcohol, tetrahydro linalool, dipropylene glycol, hexylene glycol, p-tert-butyl cyclohexanol, phenyl hexanol, 3,3,5-trimethyl hexanol, cis-trimethyl cyclohexanol, trans-2-hexanol, bornyl methoxy cyclohexanol, ethylene tridecane dioate, amyl salicylate, dimethyl benzyl carbinyl acetate, fenchyl acetate, n-hexyl salicylate, iso-bornyl acetate, o-tert-butyl cyclohexyl acetate, p-tert-butyl cyclohexyl acetate, phenyl ethyl acetate, tricyclodecenyl acetate, styrallyl acetate, methyl salicylate, allylamyl glycolate, allyl capronate, ethyl capronate, ethyl cinnamate, geranyl acetate, n-hexyl acetate, iso-amyl acetate, iso-nonyl acetate, triethyl citrate, rosephenene, dihydro mycenyl acetate, butyl-2-methyl varerate, trans-2-hexenyl acetate, dl-camphor, coumarin, pentyl cyclopentanone, iso-menthone, p-hydroxy phenyl butanone, 6-acetyl-1,1,3,4,4,6-hexamethyl tetrahydronaphthalene, ionone, p-tert-butyl cyclohexanone, o-tert-butyl cyclohexanone, 1-(2,6,6-trimethyl-1,3-cyclohexadiene-1-yl)-2-butene-1-one(Damascenone), 1-(2,6,6-trimethyl-2-cyclohexene 1-yl)-2-butene-1-one(Damascone .alpha.), 1-(2,6,6-trimethyl-1-cyclohexene-1-yl)-2-butene-1-one(Damascone beta.), 1-(2,6,6-trimethyl-3-cyclohexene-1-yl)-2-butene-1-one (Delta Damascone), Dihydro Floriffone TD), p-mentha-8-thiol-3-one, gamma.-undecalactone, phenyl acetaldehyde dimethylacetal, octyl aldehyde, citronellal, methyl nonyl acetaldehyde, diphenyl oxide, phenyl ethyl isoamyl ether, 1,3,4,6,7,8-hexahydro-4,6,6,7,8,8-hexamethyl-cyclopenta-.gamma.-2-benzopyra n (Galaxolide), eucalyptus oil, lavender oil, lime oil, lemon oil, tangerine oil, orange oil, grapefruit oil, pineneedle oil, peppermint oil, rosemary oil, spearmint oil, d'limonene, benzaldehyde, gamma nonalactone, lactones (in general), ethyl-2-methyl butyrate, alcohols, esters of citronellol, geraniol, nerol or mixtures thereof.

The invention utilizes the antioxidative materials found in the Labiatae family of plants, primarily rosemary, to stop the destruction of coloring agents found in citrus, paprika, capsicum, tumeric, and the like materials that are used to provide color in their end use. The materials are extracted out and placed in a form which is conducive to the introduction in the F&F products. This invention makes use, but not limited to, the following compounds found in rosemary: carnosic acid, carnasol, rosmanol, rosmadial, and rosmarinic acid or mixtures thereof.

The stabilized materials find utility in foods, seasoning blends, personal care products, such as soaps, shampoos, fragrances, and flavors and the like.

### EXAMPLES

The invention allows for introduction of, e.g., the rosemary extract in a number of ways, however, only a direct dilution into a carrier material will be discussed herein. It has been discovered that by adding a material that contains the rosemary extract in predetermined levels to an F&F product the material will be protected against color-degradation processes. The system employed for the detection of color loss was a BYK Gardner colorimeter which has the ability to read in Hunter Lab units. Orange, Lemon, and Grapefruit single fold materials were analyzed 5 months after the addition of the protecting extract was added. Unprotected controls were also started. The materials sat in clear glass flasks in indirect sunlight in a laboratory setting. The materials were tested in 1 cm. cells. The results are set forth in the following table.

### Examples

| Materials | desired (ppm) | carrier |
|---|---|---|
| Citrus Oils | 50 to 2000 | citrus oil |
| Oleoresin Paprika | 50 to 1000 | oleoresin paprika |
| Soaps, personal care | 100 to 1000 | citrus oil, aroma chemical |
| products | | |
| Seasoning blends | 500 to 2000 | starch or oleo, or liquid |

1. 180 kg of Orange oil had 1.8 k of orange oil spiked with 5000 ppm of
the rosemary extract to give a final value on 50 ppm.
2. 5000 pounds of a seasoning blend had 50 pounds of starch added spiked
with 200000 ppm of rosemary extract added

Below is a summary of the analytical data:

| Oil | L value | % loss | a value | % loss | b value | % loss |
|---|---|---|---|---|---|---|
| Grapefruit pro- | 87.2 | — | −13.1 | — | 49.3 | — |
| tected | | | | | | |
| Grapefruit con- | 99.1 | 12.1 | −7.9 | 40 | 17.1 | 56.4 |
| trol | | | | | | |
| Lemon protect- | 89.7 | — | −16.0 | — | 47.9 | — |
| ed | | | | | | |
| Lemon control | 99.0 | 9.4 | −4.2 | 73.7 | 6.6 | 86.2 |
| Orange protect- | 84 | — | 4.5 | — | 58 | — |
| ed | | | | | | |
| Orange control | 99.6 | 15.7 | −8.4 | 64 | 15.6 | 63.2 |

1. A fragrance or flavor composition comprising a fragrance effective amount of a fragrance compound, a flavor effective amount of a flavor compound or mixture thereof and an amount of a natural antioxidant that is substantially inert with respect to said fragrance compound, flavor compound or mixture thereof in an amount sufficient to substantially stabilize said fragrance or flavor composition against color-degradation.

2. The composition of claim 1 wherein said natural antioxidant is an extract of a member of the family Labiatae or an active antioxidant ingredient thereof.

3. The composition of claim 2 wherein said Labiatae family member is rosemary.

4. The composition of claim 3 wherein said antioxidant ingredient of said rosemary extract is camosic acid, carnasol, rosmanol, rosmadial, rosmarinic acid or mixtures thereof.

5. The composition of claim 1 wherein said fragrance compound, flavor compound or mixture thereof is citral, p-cymene, borneol, cinnamic alcohol, cinnamic aldehyde, dimethyl benzyl carbinol, 1-menthol, fenchyl alcohol, phenyl ethyl alcohol, o-tert-butyl cyclohexanol, 2-sec-butyl cyclohexanol, lauryl alcohol, 2-methyl undecanol, hexyl alcohol, citronellol, dihydro myrcenol, iso-amyl alcohol, tetrahydro linalool, dipropylene glycol, hexylene glycol, p-tert-butyl cyclohexanol, phenyl hexanol, 3,3,5-trimethyl hexanol, cis-trimethyl cyclohexanol, trans-2-hexanol, bornyl methoxy cyclohexanol, ethylene tridecane dioate, amyl salicylate, dimethyl benzyl carbinyl acetate, fenchyl acetate, n-hexyl salicylate, iso-bornyl acetate, o-tert-butyl cyclohexyl acetate, p-tert-butyl cyclohexyl acetate, phenyl ethyl acetate, tricyclodecenyl acetate, styrallyl acetate, methyl salicylate, allylamyl glycolate, allyl capronate, ethyl capronate, ethyl cinnamate, geranyl acetate, n-hexyl acetate, iso-amyl acetate, iso-nonyl acetate, triethyl citrate, rosephenene, dihydro mycenyl acetate, butyl-2-methyl varerate, trans-2-hexenyl acetate, dl-camphor, coumarin, pentyl cyclopentanone, iso-menthone, p-hydroxy phenyl butanone, 6-acetyl-1,1,3,4,4,6-hexamethyl tetrahydronaphthalene, ionone, p-tert-butyl cyclohexanone, o-tert-butyl cyclohexanone, 1-(2,6,6-trimethyl-1,3-cyclohexadiene-1-yl)-2-butene-1-one(Damascenone), 1-(2,6,6-trimethyl-2-cyclohexene 1-yl)-2-butene-1-one(Damascone .alpha.), 1-(2,6,6-trimethyl-1-cyclohexene-1-yl)-2-butene-1-one(Damascone .beta.), 1-(2,6,6-trimethyl-3-cyclohexene-1-yl)-2-butene-1-one (Delta Damascone), Dihydro Floriffone TD), p-mentha-8-thiol-3-one, gamma.-undecalactone, phenyl acetaldehyde dimethylacetal, octyl aldehyde, citronellal, methyl nonyl acetaldehyde, diphenyl oxide, phenyl ethyl isoamyl ether, 1,3,4,6,7,8-hexahydro-4,6,6,7,8,8-hexamethyl-cyclopenta-.gamma.-2-benzopyra n (Galaxolide), eucalyptus oil, lavender oil, lime oil, lemon oil, tangerine oil, orange oil, grapefruit oil, pineneedle oil, peppermint oil, rosemary oil, spearmint oil, d'limonene, benzaldehyde, gamma nonalactone, lactones (in general), ethyl-2-methyl butyrate, alcohols, esters of citronellol, geraniol, nerol or mixtures thereof.

6. A method for stabilizing a fragrance or flavor composition against color-degradation, said fragrance or flavor composition comprising a fragrance effective amount of a fragrance compound, a flavor effective amount of a flavor compound or mixture thereof, said method comprising the step of incorporating a stabilizing amount of a natural antioxidant in said composition.

7. The method of claim 6 wherein said natural antioxidant is an extract of a member of the family Labiatae or an active antioxidant ingredient thereof.

8. The method of claim 7 wherein said Labiatae family member is rosemary.

9. The method of claim 8 wherein said antioxidant ingredient of said rosemary extract is carnosic acid, carnasol, rosmanol, rosmadial, rosmarinic acid or mixtures thereof.

10. The method of claim 6 wherein said fragrance compound or mixture thereof is citral, p-cymene, borneol, cinnamic alcohol, cinnamic aldehyde, dimethyl benzyl carbinol, 1-menthol, fenchyl alcohol, phenyl ethyl alcohol, o-tert-butyl cyclohexanol, 2-sec-butyl cyclohexanol, lauryl alcohol, 2-methyl undecanol, hexyl alcohol, citronellol, dihydro myrcenol, iso-amyl alcohol, tetrahydro linalool, dipropylene glycol, hexylene glycol, p-tert-butyl cyclohexanol, phenyl hexanol, 3,3,5-trimethyl hexanol, cis-trimethyl cyclohexanol, trans-2-hexanol, bornyl methoxy cyclohexanol, ethylene tridecane dioate, amyl salicylate, dimethyl benzyl carbinyl acetate, fenchyl acetate, n-hexyl salicylate, iso-bornyl acetate, o-tert-butyl cyclohexyl acetate, p-tert-butyl cyclohexyl acetate, phenyl ethyl acetate, tricyclodecenyl acetate, styrallyl acetate, methyl salicylate, allylamyl glycolate, allyl capronate, ethyl capronate, ethyl cinnamate, geranyl acetate, n-hexyl acetate, iso-amyl acetate, iso-nonyl acetate, triethyl citrate, rosephenene, dihydro mycenyl acetate, butyl-2-methyl varerate, trans-2-hexenyl acetate, dl-camphor, coumarin, pentyl cyclopentanone, iso-menthone, p-hydroxy phenyl butanone, 6-acetyl-1,1,3,4,4,6-hexamethyl tetrahydronaphthalene, ionone, p-tert-butyl cyclohexanone, o-tert-butyl cyclohexanone, 1-(2,6,6-trimethyl-1,3-cyclohexadiene-1-yl)-2-butene-11-one(Damascenone), 1-(2,6,6-trimethyl-2-cyclohexene 1-yl)-2-butene-1-one(Damascone alpha.), 1-(2,6,6-trimethyl-1-cyclohexene-1-yl)-2-butene-1-one(Damascone .beta.), 1-(2,6,6-trimethyl-3-cyclohexene-1-yl)-2-butene-1-one (Delta Damascone), Dihydro Floriffone TD), p-mentha-8-thiol-3-one, gamma.-undecalactone, phenyl acetaldehyde dimethylacetal, octyl aldehyde, citronellal, methyl nonyl acetaldehyde, diphenyl oxide, phenyl ethyl isoamyl ether, 1,3,4,6,7,8-hexahydro-4,6,6,7,8,8-hexamethyl-cyclopenta-.gamma.-2-benzopyra n (Galaxolide), eucalyptus oil, lavender oil, lime oil, lemon oil, tangerine oil, orange oil, grapefruit oil, pineneedle oil, peppermint oil, rosemary oil, spearmint oil, d'limonene, benzaldehyde, gamma nonalactone, lactones (in general), ethyl-2-methyl butyrate, alcohols, esters of citronellol, geraniol, nerol or mixtures thereof, benzaldehyde or mixtures thereof.

11. A method of using a fragrance or flavor composition, said fragrance or flavor composition comprising a fragrance effective amount of a fragrance compound, a flavor effective amount of a flavor compound or mixture thereof and an amount of a natural antioxidant that is substantially inert with respect to said fragrance compound, flavor compound or mixture thereof in an amount sufficient to substantially stabilize said fragrance or flavor composition against color degradation, comprising forming a composition containing an effective amount of said composition with a suitable carrier.

12. The method of claim 11 wherein said natural antioxidant is an extract of a member of the family Labiatae or an active antioxidant ingredient thereof.

13. The method of claim 12 wherein said Labiatae family member is rosemary.

14. The method of claim 13 wherein said antioxidant ingredient of said rosemary extract is camosic acid, carnasol, rosmanol, rosmadial, rosmarinic acid or mixtures thereof.

15. The method of claim 11 wherein said fragrance compound or mixture thereof is citral, p-cymene, borneol, cinnamic alcohol, cinnamic aldehyde, dimethyl benzyl carbinol, 1-menthol, fenchyl alcohol, phenyl ethyl alcohol, o-tert-butyl cyclohexanol, 2-sec-butyl cyclohexanol, lauryl alcohol, 2-methyl undecanol, hexyl alcohol, citronellol, dihydro myrcenol, iso-amyl alcohol, tetrahydro linalool, dipropylene glycol, hexylene glycol, p-tert-butyl cyclohexanol, phenyl hexanol, 3,3,5-trimethyl hexanol, cis-trimethyl cyclohexanol, trans-2-hexanol, bornyl methoxy cyclohexanol, ethylene tridecane dioate, amyl salicylate, dimethyl benzyl carbinyl acetate, fenchyl acetate, n-hexyl salicylate, iso-bornyl acetate, o-tert-butyl cyclohexyl acetate, p-tert-butyl cyclohexyl acetate, phenyl ethyl acetate, tricyclodecenyl acetate, styrallyl acetate, methyl salicylate, allylamyl glycolate, allyl capronate, ethyl capronate, ethyl cinnamate, geranyl acetate, n-hexyl acetate, iso-amyl acetate, iso-nonyl acetate, triethyl citrate, rosephenene, dihydro mycenyl acetate, butyl-2-methyl varerate, trans-2-hexenyl acetate, dl-camphor, coumarin, pentyl cyclopentanone, iso-menthone, p-hydroxy phenyl butanone, 6-acetyl-1, 1,3,4,4,6-hexamethyl tetrahydronaphthalene, ionone, p-tert-butyl cyclohexanone, o-tert-butyl cyclohexanone, 1-(2,6,6-trimethyl-1,3-cyclohexadiene-1-yl)-2-butene-1-one(Damascenone), 1-(2,6,6-trimethyl-2-cyclohexene 1-yl)-2-butene-1-one(Damascone .alpha.), 1-(2,6,6-trimethyl-1-cyclohexene-1-yl)-2-butene-1-one(Damascone .beta.), 1-(2,6,6-trimethyl-3-cyclohexene-1-yl)-2-butene-1-one (Delta Damascone), Dihydro Floriffone TD), p-mentha-8-thiol-3-one, gamma.-undecalactone, phenyl acetaldehyde dimethylacetal, octyl aldehyde, citronellal, methyl nonyl acetaldehyde, diphenyl oxide, phenyl ethyl isoamyl ether, 1,3,4,6,7,8-hexahydro-4,6,6,7,8,8-hexamethyl-cyclopenta-.gamma.-2-benzopyra n (Galaxolide), eucalyptus oil, lavender oil, lime oil, lemon oil, tangerine oil, orange oil, grapefruit oil, pineneedle oil, peppermint oil, rosemary oil, spearmint oil, d'limonene, benzaldehyde, gamma nonalactone, lactones (in general), ethyl-2-methyl butyrate, alcohols, esters of citronellol, geraniol, nerol or mixtures thereof.

16. The method of claim 11 wherein said carrier is a food, cosmetic product, prepared food, meat, seasoning or flavoring blend.

17. An article of manufacture comprising a carrier having a fragrance or flavor composition incorporated therein wherein said fragrance or flavor composition comprises a fragrance effective amount of a fragrance compound, a flavor effective amount of a flavor compound or mixture thereof and an amount of a natural antioxidant that is substantially inert with respect to said fragrance compound, flavor compound or mixture thereof in an amount sufficient to substantially stabilize said fragrance or flavor composition against color degradation.

18. The article of manufacture of claim 17 wherein said natural antioxidant is an extract of a member of the family Labiatae or an active antioxidant ingredient thereof.

19. The article of manufacture of claim 18 wherein said Labiatae family member is rosemary.

20. The article of manufacture of claim 19 wherein said antioxidant ingredient of said rosemary extract is carnosic acid, carnasol, rosmanol, rosmadial, rosmarinic acid or mixtures thereof.

21. The article of manufacture of claim 17 wherein said fragrance compound or mixture thereof is citral, p-cymene, borneol, cinnamic alcohol, cinnamic aldehyde, dimethyl benzyl carbinol, 1-menthol, fenchyl alcohol, phenyl ethyl alcohol, o-tert-butyl cyclohexanol, 2-sec-butyl cyclohexanol, lauryl alcohol, 2-methyl undecanol, hexyl alcohol, citronellol, dihydro myrcenol, iso-amyl alcohol, tetrahydro linalool, dipropylene glycol, hexylene glycol, p-tert-butyl cyclohexanol, phenyl hexanol, 3,3,5-trimethyl hexanol, cis-trimethyl cyclohexanol, trans-2-hexanol, bornyl methoxy cyclohexanol, ethylene tridecane dioate, amyl salicylate, dimethyl benzyl carbinyl acetate, fenchyl acetate, n-hexyl salicylate, iso-bornyl acetate, o-tert-butyl cyclohexyl acetate, p-tert-butyl cyclohexyl acetate, phenyl ethyl acetate, tricyclodecenyl acetate, styrallyl acetate, methyl salicylate, allylamyl glycolate, allyl capronate, ethyl capronate, ethyl cinnamate, geranyl acetate, n-hexyl acetate, iso-amyl acetate, iso-nonyl acetate, triethyl citrate, rosephenene, dihydro mycenyl acetate, butyl-2-methyl varerate, trans-2-hexenyl acetate, dl-camphor, coumarin, pentyl cyclopentanone, iso-menthone, p-hydroxy phenyl butanone, 6-acetyl-1,1,3,4,4,6-hexamethyl tetrahydronaphthalene, ionone, p-tert-butyl cyclohexanone, o-tert-butyl cyclohexanone, 1-(2,6,6-trimethyl-1,3-cyclohexadiene-1-yl)-2-butene-1-one(Damascenone), 1-(2,6,6-trimethyl-2-cyclohexene 1-yl)-2-butene-1-one(Damascone .alpha.), 1-(2,6,6-trimethyl-1-cyclohexene-1-yl)-2-butene-1-one(Damascone .beta.), 1-(2,6,6-trimethyl-3-cyclohexene-1-yl)-2-butene-1-one (Delta Damascone), Dihydro Floriffone TD), p-mentha-8-thiol-3-one, gamma.-undecalactone, phenyl acetaldehyde dimethylacetal, octyl aldehyde, citronellal, methyl nonyl acetaldehyde, diphenyl oxide, phenyl ethyl isoamyl ether, 1,3,4,6,7,8-hexahydro-4,6,6,7,8,8-hexamethyl-cyclopenta-.gamma.-2-benzopyra n (Galaxolide), eucalyptus oil, lavender oil, lime oil, lemon oil, tangerine oil, orange oil, grapefruit oil, pineneedle oil, peppermint oil, rosemary oil, spearmint oil, d'limonene, benzaldehyde, gamma nonalactone, lactones (in general), ethyl-2-methyl butyrate, alcohols, esters of citronellol, geraniol, nerol or mixtures thereof.

22. The article of manufacture of claim 21 wherein said carrier is a food, cosmetic product, prepared food, meat, seasoning or flavoring blend.
